(19) United States
(12) Patent Application Publication
(10) Pub. No.: US 2022/0265407 A1
(43) Pub. Date: Aug. 25, 2022

(54) ARRANGEMENTS FOR CAPTURING AEROSOL DURING DENTAL PROCEDURES AND METHODS

(71) Applicant: Donaldson Company, Inc., Bloomington, MN (US)

(72) Inventors: Timothy H. Grafe, Edina, MN (US); Qinghui Yuan, Edina, MN (US); Stephen J. Lawton, Charminster (GB); Richard J. Bloomfield, Poole (GB)

(21) Appl. No.: 17/667,847

(22) Filed: Feb. 9, 2022

Related U.S. Application Data
(60) Provisional application No. 63/149,485, filed on Feb. 15, 2021.

(51) Int. Cl. A61C 17/08; A61G 15/16; A41D 13/11

(52) U.S. Cl. CPC A61C 17/08; A61G 15/16; A41D 13/1184

(57) ABSTRACT

Arrangements for capturing aerosol during dental procedures include an extraoral suction device and a blower arrangement to induce an airflow zone to push aerosol to the suction device. Blower arrangements can include plenums in the form of a tube, or a face mask, or attached to safety glasses. Extraoral suction devices can be wearable in the form of appliances to be fitted onto either the patient or the dental professional. Improved extraoral suction devices can include a screen secured to a nozzle of the device.

### CROSS-REFERENCE TO RELATED APPLICATIONS

This application claims the benefit of U.S. Provisional Application No. 63/149,485 filed Feb. 15, 2021, which is incorporated herein by reference in its entirety.

### TECHNICAL FIELD

This disclosure relates to arrangements and methods for improving recapture of aerosol during dental procedures. In particular, this disclosure relates to arrangements and methods of using one or more of extraoral suction devices; blower arrangements with extraoral suction devices; and/or wearable appliances.

### BACKGROUND

Dental professionals and dental patients need protection for aerosol generation procedures during dental care. The need for this protection is heighted currently while viruses, such as Covid-19, are prevalent.

In the past, there have been used extraoral suction devices, sometimes called local extractors, which can suck aerosol into a filtration system, thus reducing the contaminants from the environment. One example of an extraoral suction device is described in U.S. Pat. No. 9,131,997, incorporated herein by reference.

In some extraoral suction devices, the suction is ineffective to deal with dynamic dental operation procedures and various room conditions. Because of this, the capture efficiency of the aerosol and any viruses within the aerosol is not guaranteed. With no guarantee of aerosol or virus captures efficiency, it is hard to justify an investment in this equipment.

What is needed is an improvement to the capture efficiency of aerosols and viruses, without creating additional discomfort to the patient. An arrangement that improves the robustness of capture efficiency under various conditions, as well as maintaining a comfort level for the patient, is desirable.

### SUMMARY

In one aspect, an arrangement for capturing aerosol during a dental procedure on a human is provided. The arrangement includes an extraoral suction device; and a blower arrangement to induce an airflow zone to push aerosol to the suction device.

In some arrangements, the blower arrangement comprises an airflow plenum with a plurality of holes sized to permit a flow of air from the plenum and toward the suction device.

The plenum may comprise a ring sized to surround a human head from the crown of the head to under a chin and back to the crown.

In some examples, the plenum is a tube, and the holes are in a front of the tube facing away from the human head.

In one or more embodiments, the holes are circumferentially spaced along the tube. In some examples, they may be evenly circumferentially spaced, but many variations are possible.

In some example embodiments, the plenum comprises a face mask to cover a human face. The mask has an opening arrangement located to expose a mouth and nostrils, when the mask is over a human face. The holes are along an outer periphery of the mask and oriented to direct airflow from the holes toward the opening arrangement.

In one or more example embodiments, the plenum is attached to safety glasses worn over the eyes, and the holes are oriented to direct airflow away from the eyes and toward a mouth of the human.

The blower arrangement has a positive pressure side for inducing the air flow zone pushing aerosol to the suction device, and in some embodiments, the negative pressure side is used for inducing a vacuum pressure in the suction device.

In some preferred arrangements, the blower arrangement is configured to produce the air flow zone to flow from an upper position to a lower position.

One or more embodiments can further include an overhead light arrangement, in which the air flow zone flows from a duct attached to the overhead light arrangement.

In another aspect, an extraoral suction device is provided including a hose connected to a vacuum; a nozzle at the end of the hose; and a screen secured to the nozzle.

In some examples, the screen extends from an outer rim of the nozzle less than 270°.

In many examples, the screen is transparent.

In another aspect, a wearable extraoral suction device for capturing aerosol during a dental procedure is provided. The wearable extraoral suction device includes an appliance to be fitted onto a human; a hose connected to a vacuum secured to the appliance; and one or more ducts within the appliance and connected to the hose.

In some examples, the appliance is a flexible mitten sized to fit over a gloved hand of a user.

In some examples, the appliance is a visor worn by a patient.

In another aspect, an arrangement in a dental office for capturing aerosol during a dental procedure on a human is provided. The arrangement includes a dental chair; and an extraoral suction device in a vicinity of the dental chair to draw in aerosol from a patient in the dental chair.

The extraoral suction device can be arranged to draw in aerosol from an induced air flow zone.

In some cases, there further includes an overhead light arrangement, and wherein the air flow zone flows from a duct attached to the overhead light arrangement.

In some embodiments, there further includes a blower arrangement to induce the air flow zone.

The extraoral suction device can be attached to the dental chair.

The extraoral suction device can be part of a cabinet in the dental office; part of furniture in the dental office; and/or part of fixtures in the dental office.

The liner may comprise a filtration material, in some examples.

In some examples, the liner can have an antimicrobial treatment.

In one or more embodiments, the extraoral suction device is in accordance with various examples above.

A variety examples of desirable product features or methods are set forth in the description that follows, and in part, will be apparent from the description, or may be learned by various aspects of this disclosure. The aspects of this disclosure may relate to individual features, as well as combinations of features. It is to be understood that both the foregoing detailed description are explanatory only and are not restrictive of the claimed inventions.

### BRIEF DESCRIPTION OF THE DRAWINGS

FIG. 1 is a perspective view of an example prior art extraoral extractor in a typical environment of use;

FIG. 2 is a schematic diagram of concepts usable consistent with principles of this disclosure;

FIG. 3 is a schematic diagram showing an example embodiment, constructed in accordance with principles of this disclosure;

FIG. 4 is another schematic diagram of the embodiment of FIG. 3;

FIG. 5 is a schematic illustration of another embodiment, constructed in accordance with principles of this disclosure;

FIG. 6 is a perspective view of the embodiment of FIG. 5;

FIG. 7 is a schematic diagram of another embodiment, constructed in accordance with principles of this disclosure;

FIG. 8 is a top plan view of the embodiment of FIG. 7;

FIG. 9 is a schematic diagram showing a simulation, in which the embodiment of FIGS. 3-4 are not operating;

FIG. 10 is another schematic diagram showing a simulation, in which the embodiment of FIGS. 3-4 are not operating;

FIG. 11 is another schematic diagram showing a simulation, this time with the use of the embodiment of FIGS. 3-4 in operation;

FIG. 12 is a schematic, perspective view of another embodiment, constructed in accordance with principles of this disclosure;

FIG. 13 is a schematic, perspective view of another embodiment, constructed in accordance with principles of this disclosure;

FIG. 14 is a schematic, perspective view of another embodiment, constructed in accordance with principles of this disclosure; and

FIG. 15 is another embodiment, similar to that shown in FIG. 14, constructed in accordance with principles of this disclosure;

FIG. 16 is a perspective view of a nozzle of an extra oral extractor having an insertable and disposable liner;

FIG. 17 is a top plan view of the liner used in the nozzle of FIG. 16;

FIG. 18 is a top plan view of another embodiment of a liner, this embodiment being a double layer;

FIG. 19 is a perspective view of a nozzle of an extra oral extractor including a sock liner;

FIG. 20 is an end view of the embodiment of FIG. 19;

FIG. 21 is a perspective view of a truncated, open ended sock liner for use with the nozzle of an extra oral extractor;

FIG. 22 is a perspective view of the truncated sock liner of FIG. 21 secured onto the extra oral extractor and secured with an elastic band;

FIG. 23 is another perspective view of the embodiment of FIG. 22;

FIG. 24 is a top plan view of a fabric sock for use with an extra oral extractor having a double layer; and

FIG. 25 is another view of the fabric sock of FIG. 24.

### DETAILED DESCRIPTION

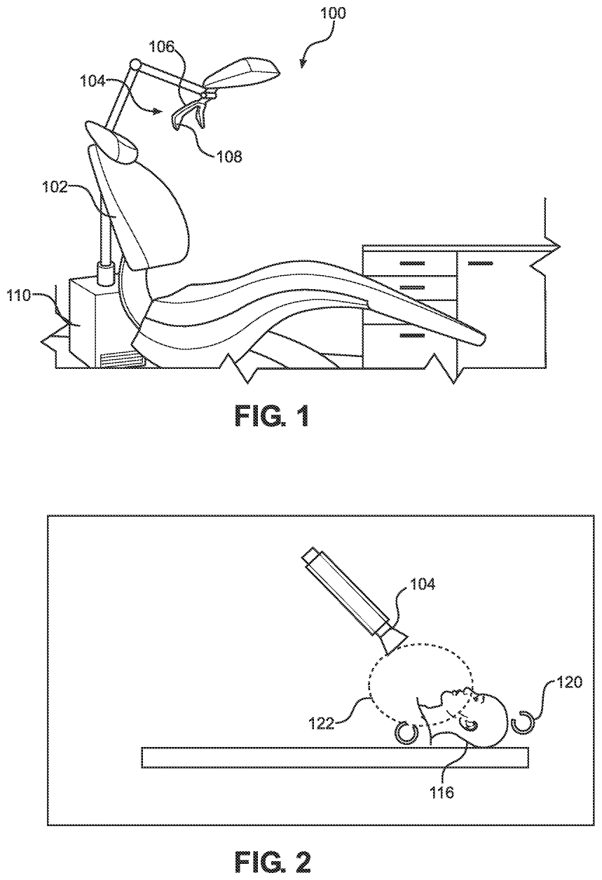

FIG. 1 illustrates a typical environment of use for principles of this disclosure. In FIG. 1 is a typical dental office 100 including a chair 102, in which the patient is seated. An extraoral extractor 104 can be seen located in proximity to the head area of the chair 102. The extraoral extractor 104 includes a suction device 106 having a nozzle 108. The suction device 106 is connected to a vacuum inducing generator 110. The vacuum inducing generator 110 produces a vacuum or suction, which is present at the nozzle 108, which ideally will draw in aerosol generated during a dental procedure. The aerosol can include viruses, such as Covid-19.

The extraoral extractor 104 can be inefficient in its capture of aerosol due to various conditions in the dental office 100, such as room size, ventilation, equipment operation, and human factors.

To improve capture efficiency, additional airflow is introduced beyond the suction port of the suction device 106, to create an airflow zone to move all of the particles and generated aerosol into the extraoral extractor 104.

In reference now to FIG. 2, a schematic diagram illustrates one example concept of improvements to the prior art. In the diagram of FIG. 2, the head of a patient can be seen at 116. Extending over and spaced from the mouth of the patient 116 is extraoral suction device 106. A blower arrangement 120 is shown schematically at the crown of the patients head 116 and under the chin of the patients head 116. The blower arrangement 120 pushes out an airflow to induce an airflow zone shown in broken lines at 122, which will push aerosol to the suction device 106. The blower arrangement 120 surrounds the mouth and nose of the patient 116, to push out air, creating the airflow zone 122.

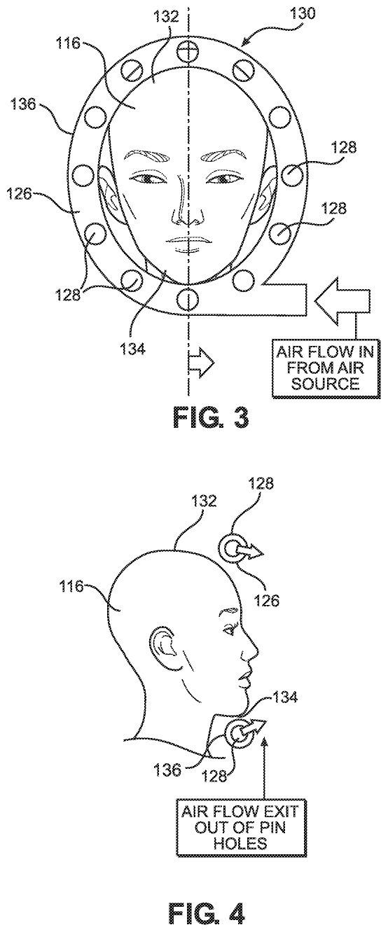

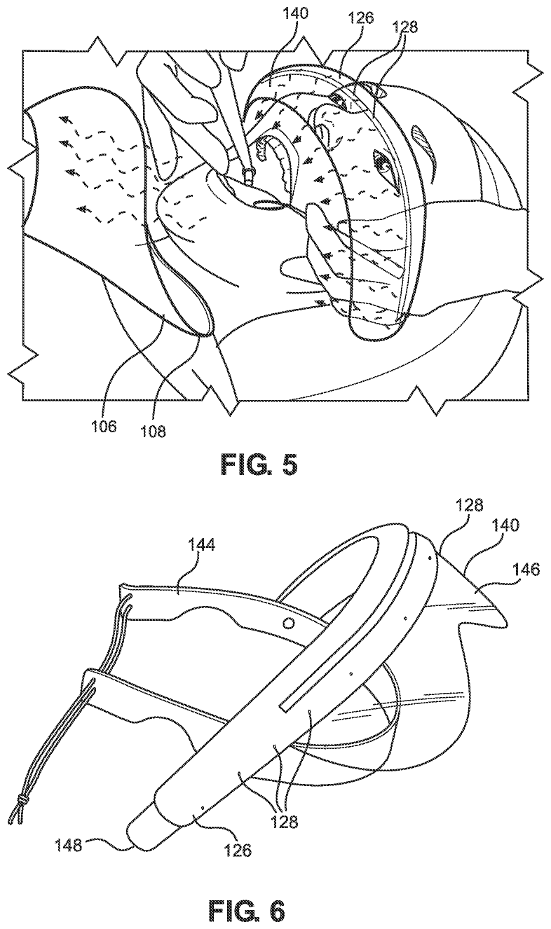

In FIGS. 3 and 4, the blower arrangement 120 includes an airflow plenum 126 with a plurality of holes 128 sized to permit a flow of air from the plenum 126 and toward the suction device 106. The holes 128 can be very small, such as pinholes. Many variations are possible, including varying the size of the holes 128. One of the objections in some example embodiments aims for an even flow rate around the entire device, which can be achieved if the holes 128 are different sizes, with smaller holes 128 being near the point where the air is pumped in. As the holes 128 get farther away from the air source, they would be larger. In general, the size of the holes 128 will depend on the flow rated from the blower arrangement 120. The blower flow rate can be decoupled from the suction flow rate, which means that the total flow rate out of all of the holes 128 can be different from the suction flow rate. Typically, it is desirable to have a smaller blower flow rate to have a quieter and more energy efficient system. In preferred systems, the flow rate of the blower arrangement 120 is determined independently, and the size(s) of the holes 128 are selected based on the flow rate of the blower arrangement 120.

In the example shown in FIGS. 3 and 4, the plenum 126 forms a ring 130 which is sized to surround the head 116 of the patient from the top or crown 132 of the head 116 down under the chin 134 and back the crown 132. The plenum 126 can be in a form of a tube 136, with the holes 128 in a front of the tube 136 facing away from the head 116. In this arrangement, airflow through the holes 128 will flow in a direction away from the head 116 into the zone 126 and into the suction device 106.

As can be seen in the example of FIG. 3, the holes 128 can be made to be evenly circumferentially spaced along the tube 136. In other embodiments, the holes 128 can be arranged in other patterns.

The holes 128 around the head 116 create the stable airflow that will naturally go to the suction device 106. This will create a very stable flow zone 122 around the head 116 to carry contaminant to the nozzle 108, even if there are disturbances in the airflow of the dental office. The airflow through the holes 128 can be independently controlled and can be smaller than the airflow of suction on the suction device 106.

In the embodiment of FIGS. 3 and 4, the tube 136 can be installed directly on the dental chair 102, or it may be in a different form, such as a headset worn by the patient. The air source can be from the extraoral suction device 106, or from a separate air pump, or from the dental chair 102. The airflow rate can be adjustable. Many variations are possible.

The blower arrangement 120 induces the airflow zone on the positive pressure side to push aerosol to the suction device 106. In some embodiments, the negative pressure side of the blower arrangement is used for inducing a vacuum pressure in the suction device 106.

In some preferred arrangements, the blower arrangement 120 is configured to produce the air flow zone to flow from an upper position to a lower position, working with gravity to help move very large droplets to the suction device 106. Very large droplets (i.e., splatter) can be more difficult to capture, due to their mass. But also due to their mass, these very large droplets do not linger in the air and pose as big of a risk to remaining airborne. Therefore this pushed air would work with gravity to move the very large droplets to the suction device 106.

In FIG. 1, the dental office 100 includes an overhead light arrangement 109. In some embodiments, the air flow zone can be configured to flow from a duct attached to the overhead light arrangement 109. Many alternatives are possible.

Another embodiment is illustrated in FIGS. 5 and 6. In this embodiment, the plenum 126 is attached to safety glasses or goggles 140. The safety glasses 140 are worn by the patient 116 to cover the eyes and protect the eyes from particulate and debris during a dental proceeding. In FIG. 5, the nozzle 108 is placed close to the chin of the patient 116, and the air holes 128 are oriented to direct airflow away from the eyes and toward a mouth and toward the nozzle 108. An air slot can be designed using the Coanda effect to direct air toward the nozzle 108 and away from the dental professional.

In FIG. 6, the safety glasses 140 are shown including an elastic headband 144. The safety glasses 140 can include a hinged visor 146 having a hose connection 148, to hook up to a hose with an air source. The visor 146 can be removable and replaceable.

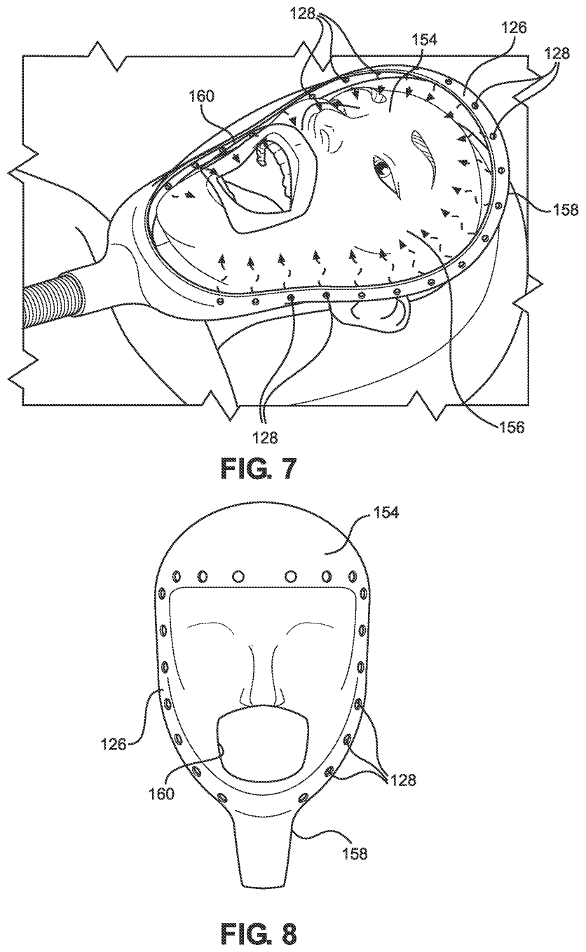

FIGS. 7 and 8 show another embodiment in which the plenum 126 comprises a face mask 154. The face mask 154 is sized to cover a human face 156. The mask 154 has an outer periphery 158 which is generally along an outer boarder of the face 156.

The mask 154 has an opening arrangement 160 located to expose the mouth and nostrils of the face 156, when the mask is over the face 156. In FIG. 8, the opening arrangement 160 is shown as a single opening, but the opening arrangement 160 could be more than one opening, such as shown FIG. 7 where there is a separate opening for the mouth and a separate opening for the nostrils.

Holes 128 for the airflow are along the periphery 158 and are oriented to direct airflow from the holes 128 and toward the opening arrangement 160. From there, it flows into the airflow zone 122 and into the suction device 106.

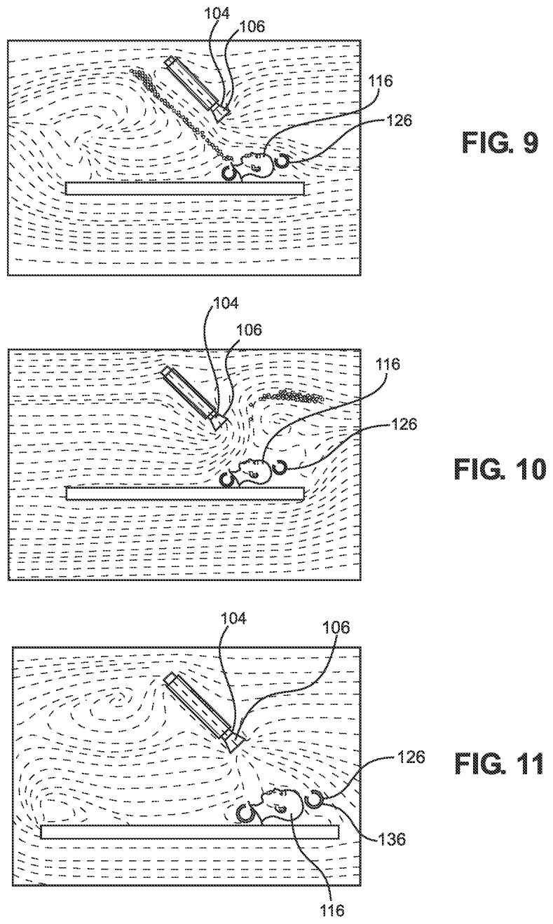

The diagrams of FIGS. 9-11 show the effectiveness of the embodiments of FIGS. 2-8. When there are airflow disturbances in the dental office, the suction zone created by an extraoral extractor 104 is distorted. In FIG. 9, aerosol and droplets is simulated to eject out of a patients mouth, with an air disturbance to the left. In FIG. 9, only the extraoral extractor 104, is used, and while the plenum 126 is shown in the diagram, in this simulation, it is not operating. In this case, as can be seen in FIG. 9, all of the aerosol flows to the side of the extraoral extractor 104. The capture efficiency is 0%, meaning that the extraoral extractor 104 does not provide any value in capturing contaminants.

In the diagram of FIG. 10, the conditions are the same as FIG. 9 (i.e., the embodiments of FIGS. 2-8 are not operating, and only the extraoral extractor 104 is being used), but the air disturbance is to the right, which causes all of the aerosol to go to the side of the extractor 104. The capture efficiency is 0%, meaning the extraoral extractor 104 does not provide any value to capturing contaminants.

In FIG. 11, the embodiments of FIGS. 3-4 are in operation creating the stable airflow zone 122, in combination with the extraoral extractor 104. The exact same conditions as shown in FIG. 10 are created, with an air disturbance to the right. In this case, a 93% capture efficiency is demonstrated with the significant improvement from the baseline of 0%. Only a very limited portion of large droplets is left on the lips of the patient.

Other advantages are realized, including being able to set the suction flow rate to be only 50% of the full flow rate in cases that do not use one of the embodiments of FIGS. 2-8. This also lowers the noise level and lowers energy consumption. There are no obstacles for the dental procedure and no uncomfortable inserts into the mouth of the patient. The embodiments of FIGS. 2-8 maintain the performance, even for lower suction airflow, leading to a quiet procedure and lower energy consumption.

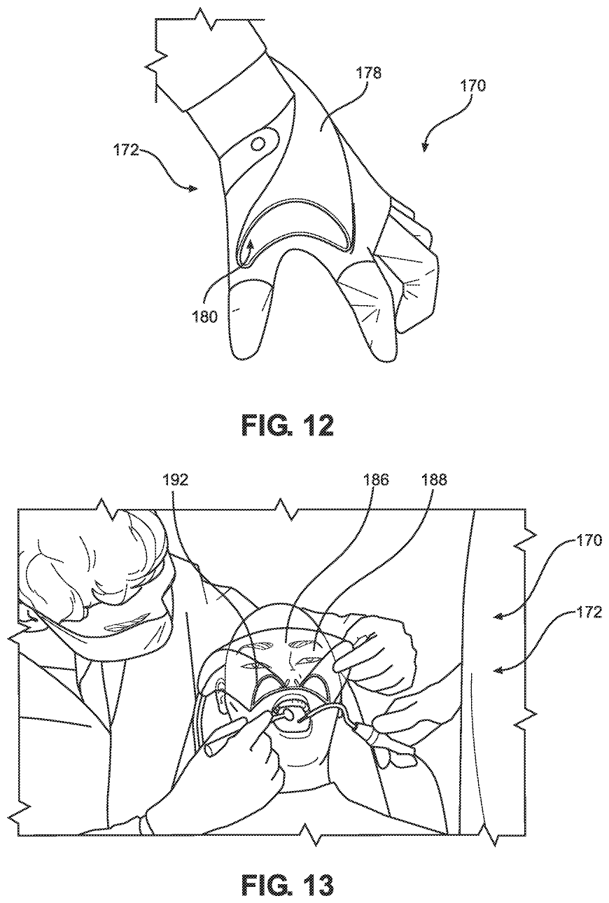

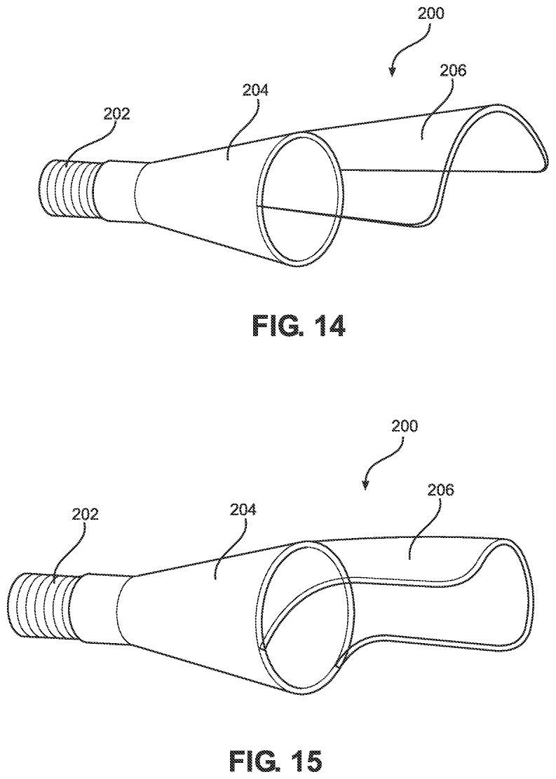

Additional embodiments are shown in FIGS. 12 and 13. In the embodiment of FIGS. 12 and 13, a wearable extraoral suction device 170 is provided. The wearable extraoral suction device 170 includes an appliance 172 to be fitted onto a human. The appliance 172 can be fitted onto either the patient or the dental professional working on the patient. In general, a hose will be provided, which is connected to a vacuum source, which is secured to the appliance 172. There will be one or more ducts within the appliance 172 and connected to the hose.

In the example of FIG. 12, the appliance 172 is a flexible mitten 178. The mitten 178 is sized to fit over a gloved hand of a user, the user being the dental professional. The mitten 178 brings the extraction point as close to the source of the aerosol as possible. The mitten 178 can be made from a variety of materials, such as molded in silicone or a similar material that is flexible and non-porous. Silicone is resistant to most chemicals and is easily cleaned.

The mitten 178 can be secured by a simple toggle and can be designed to be usable with either a right hand or a left hand.

The mitten 178 would be attachable to a flexible hose, and the hose will not deform under vacuum and should provide for ease of movement without adding excessive weight that may hinder the dental professional.

Within the mitten 178 are molded ducts 180, which will take in the aerosol from the mouth of the patient.

In FIG. 13, the appliance 172 is a visor 186 worn by the patient. The visor 186 includes a lightweight, transparent extraction funnel 188 having ducts 190 and is worn over the upper part of the face of the patient. The visor 186 can be molded so as to fit a wide variety of faces, with cheek and forehead pads for comfort. The extraction system can connect above the head of the patient at either side. The visor 186 can be incorporated into a design that would replace the usual safety glasses.

FIGS. 14 and 15 show another embodiment. In FIGS. 14 and 15 an extraoral suction device is illustrated at 200. The extraoral suction device 200 includes a hose 202, connected to a vacuum source. A nozzle 204 is at an end of the hose 202. A screen 206 is secured to the nozzle 204.

The screen 206 can extend from an outer rim of the nozzle 204 and be along the circumferential edge less than a full 360°, such as less than 270°. In some cases, the screen 206 is 180°, or less.

The screen 206 is preferably transparent, such that the dental professional can see through it. The screen 206 can be made from a variety of materials including, for example, acetate. The screen 206 will partially enclose the aerosol and limit the escape of droplets toward the dental professional.

FIG. 15 is a contoured version of the screen 206 which has better ergonomics.

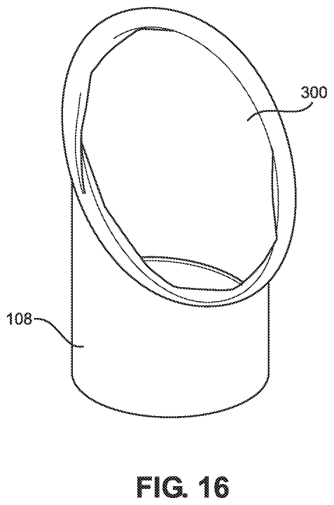

In reference now to FIGS. 16-25, it has been found that when dental hygienists use the extra oral extractor 104, the nozzle 108 can be covered in water, saliva, and blood from various oral procedures, including ultrasonic scaling and laser root planing. Without the use of an extra oral extractor, these fluids would remain airborne or settle on the patient, hygienist, or other nearby surfaces. Because these fluids are settling on the nozzle 108, rather than wiping them down, it is envisioned that a disposable liner fitting within the nozzle 108 would be helpful. FIGS. 16-25 show various embodiments of a disposable liner 300.

The disposable liner 300 can be made of a paper or a fabric material formed to line an interior of the nozzle 108 of the extra oral suction device 104. The paper can include anti-microbial coatings.

FIG. 16 shows nozzle 108 having a liner 300 lining the interior of the nozzle 108.

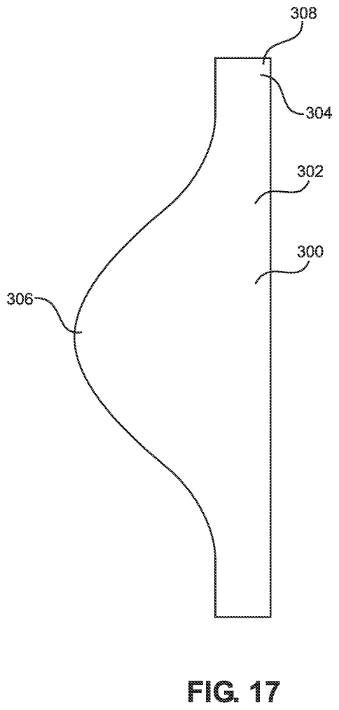

In FIG. 17, the liner 300 is shown as a paper liner made of a single layer 302. The liner 300 is provided as a flat cutout having a band 304 and a hump 306 projecting from the band 304. The band 304 can connect to itself after the liner 300 is formed into a cylinder shape. The band 304 can have a tab 308 that connects, either in the form of adhesive or by mechanical interlock. In this embodiment, the liner 300 is fitted within the interior of the nozzle 108, and it can be free of physical connection with the nozzle 108.

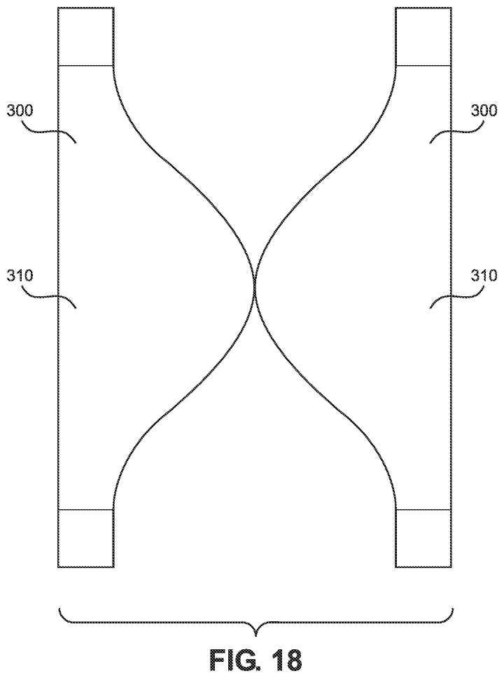

FIG. 18 shows a double layer paper liner 310. The double layer liner 310 is similar to the single layer of FIG. 17, except that it is two layers instead of one.

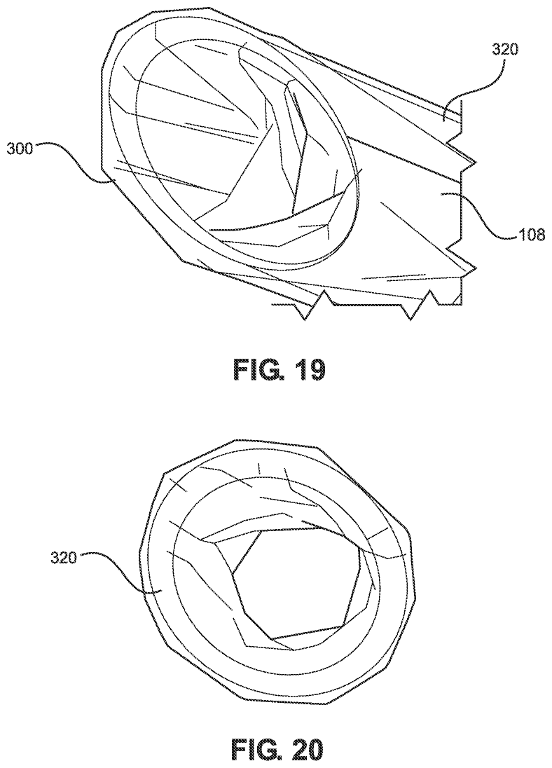

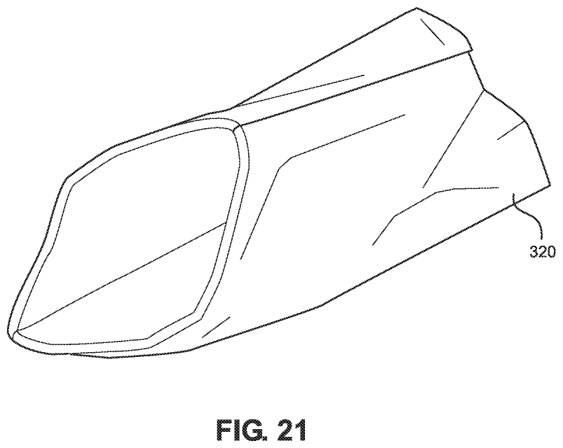

FIG. 19 illustrates a sock liner 320 made of a fabric material. The sock liner 320 is constructed and arranged to cover both the interior of the nozzle 108 and the exterior of the nozzle 108. It is secured to the nozzle 108 with an elastic or rubber band around the nozzle 108. FIG. 20 shows an end view of the nozzle 108 having the sock liner 320. FIG. 21 shows a truncated sock liner 320 before securing it to the nozzle 108.

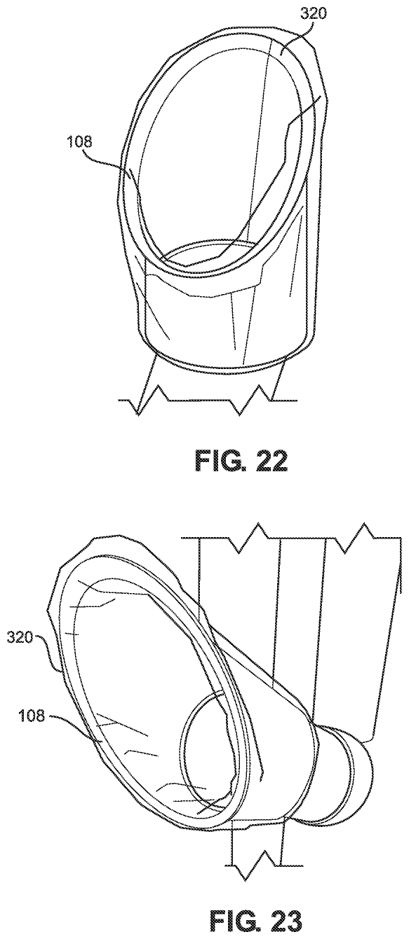

FIGS. 22 and 23 show the truncated sock liner 320 of FIG. 21 secured to the nozzle 108 with an elastic band around the nozzle 108.

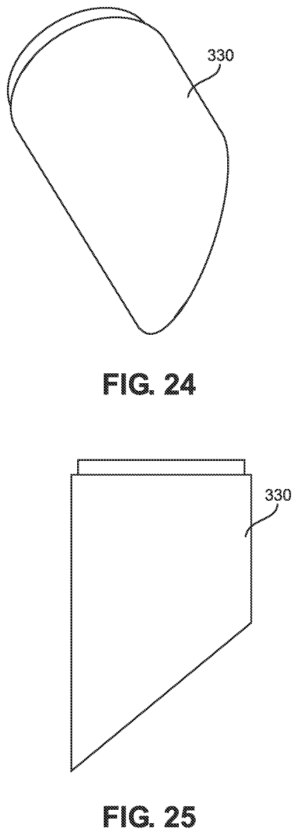

FIGS. 24 and 25 show a fabric sock at 330. The fabric sock 330 can be made of a material similar to face mask material. It can have a double layer to cover the nozzle 108 both along the interior and the exterior. The sock 330 can include fused or welded seems. It is shaped to fit the nozzle shape 108.

The liner 320 can be made from a variety of materials, including a filtration material or media. It can also have an antimicrobial treatment.

The above represents example principles. Many embodiments can be made using these principles.

1. An arrangement for capturing aerosol during a dental procedure on a human; the arrangement comprising:
(a) an extraoral suction device; and
(b) a blower arrangement to induce an air flow zone to push aerosol to the suction device.

2. The arrangement of claim 1 wherein the blower arrangement comprises an airflow plenum with a plurality of holes sized to permit a flow of air from the plenum and toward the suction device.

3. The arrangement of claim 2 wherein the plenum comprises a ring sized to surround a human head from crown of the head, to under a chin, and back to the crown.

4. The arrangement of claim 3 wherein the plenum is a tube, and the holes are in a front of the tube facing away from the human head.

5. The arrangement of claim 4 wherein the holes are circumferentially spaced along the tube.

6. The arrangement of claim 2 wherein the plenum comprises a face mask sized to cover a human face; the mask having an opening arrangement located to expose a mouth and nostrils, when the mask is over a human face; the holes being along an outer periphery of the mask and oriented to direct airflow from the holes toward the opening arrangement.

7. The arrangement of claim 2 wherein the plenum is attached to safety glasses worn over eyes, and the holes are oriented to direct airflow away from the eyes and toward a mouth of the human.

8. The arrangement of claim 1 wherein the blower arrangement has a positive pressure side for inducing the air flow zone pushing aerosol to the suction device, and a negative pressure side for inducing a vacuum pressure in the suction device.

9. The arrangement of claim 1 wherein the blower arrangement is configured to produce the air flow zone to flow from an upper position to a lower position.

10. The arrangement of claim 1 further including an overhead light arrangement, and wherein the air flow zone flows from a duct attached to the overhead light arrangement.

11. An extraoral suction device comprising:
(a) a hose connected to a vacuum;
(b) a nozzle at an end of the hose; and
(c) a screen secured to the nozzle.

12. The extraoral suction device of claim 11 wherein the screen extends from an outer rim of the nozzle less than 270 degrees.

13. The extraoral suction device of claim 11 wherein the screen is transparent.

14. The extraoral suction device of claim 11 further including a disposable liner positioned in the nozzle.

15. A wearable extraoral suction device for capturing aerosol during a dental procedure comprising:
(a) an appliance to be fitted onto a human;
(b) a hose connected to a vacuum secured to the appliance; and
(c) one or more ducts within the appliance and connected to the hose.

16. The wearable extraoral suction device of claim 15 wherein the appliance is a flexible mitten sized to fit over a gloved hand of a user.

17. The wearable extraoral suction device of claim 15 wherein the appliance is a visor worn by a patient.

18. An arrangement in a dental office for capturing aerosol during a dental procedure on a human; the arrangement comprising:
a dental chair; and
an extraoral suction device in a vicinity of the dental chair to draw in aerosol from a patient in the dental chair.

19. The arrangement of claim 18 wherein the extraoral suction device is arranged to draw in aerosol from an induced air flow zone.

20. The arrangement of claim 19 further including an overhead light arrangement, and wherein the air flow zone flows from a duct attached to the overhead light arrangement.

21. The arrangement of claim 20 further including a blower arrangement to induce the air flow zone.

22. The arrangement of claim 18 wherein the extraoral suction device is attached to at least one of: the dental chair; part of a cabinet in the dental office; part of furniture in the dental office; or part of fixtures in the dental office.

23.-33. (canceled)
